(19) United States
(12) Patent Application Publication
(10) Pub. No.: US 2024/0307099 A1
(43) Pub. Date: Sep. 19, 2024

(54) Maxillofacial Fixation Implants Having a Registration Member Relative to the Anterior Nares and Anterior Nasal Spine

(71) Applicant: Travis Simpson, Jacksonville, FL (US)

(72) Inventor: Travis Simpson, Jacksonville, FL (US)

(21) Appl. No.: 18/671,360

(22) Filed: May 22, 2024

Related U.S. Application Data
(63) Continuation of application No. 17/724,201, filed on Apr. 19, 2022.
(60) Provisional application No. 63/177,024, filed on Apr. 20, 2021.

(51) Int. Cl. A61B 17/80; A61C 5/00

(52) U.S. Cl. CPC A61B 17/8061; A61B 17/808; A61C 5/007

(57) ABSTRACT

A maxillofacial fixation implant having a registration member positioned so as to abut, surround and/or engage with the anterior nares and the anterior nasal spine of a human skull, and the method of designing, manufacturing and utilizing the implant, wherein the implant has an upper anchor implant portion adapted for affixation to the upper portion of a maxilla and a lower anchor implant portion adapted for affixation to separated bone segments of the maxilla.

### BACKGROUND OF THE INVENTION

This invention relates generally to the field of maxillofacial fixation implants and related surgical techniques, such as maxillofacial orthognathic surgical implants used in the correction of malformations of the maxilla and/or mandible and implants used to repair or reconstruct damaged bones due to trauma or the like. The invention more particularly relates to such implants and techniques wherein separated bone segments are realigned, such as for example in orthognathic surgery wherein a lower portion of the maxilla is surgically separated from the upper portion of the maxilla, such that the lower portion is realigned relative to the upper portion to produce a more desirable facial configuration. Even more particularly, the invention relates to such implants and techniques wherein maxillofacial implants are customized to better conform to the surface configuration of the maxilla, the customization utilizing computer-aided 3-D imaging to determine the optimal size and configurations for the implants.

For ease of discussion and without meant to be limiting, the implants and techniques shall be described herein with particular regard for orthognathic surgery, but it is to be understood that implants and techniques for non-orthognathic procedures using computer-assisted 3-D imaging, such as repair or reconstruction of non-surgically separated bone segments or fragments, are also included in the scope and definition of the invention.

The term orthognathic comes from the Greek words “orthos” meaning straighten and “gnathic” meaning of or related to the jaw. Orthognathic surgery may be indicated in order to make biting and chewing easier and improve chewing overall, correct problems with swallowing or speech, minimize excessive wear and breakdown of the teeth, correct bite fit or jaw closure issues, correct facial imbalance (asymmetry), such as small chins, underbites, overbites and cross bites, improve the ability of the lips to fully close comfortably, relieve pain caused by temporomandibular joint (TMJ) disorder and other jaw problems, repair facial injury or birth defects, or provide relief for obstructive sleep apnea.

In a basic maxillary orthognathic procedure, the surgeon determines the desired post-operative configuration for the maxilla, performs an osteotomy to separate a lower portion of the maxilla from the upper portion, repositions the lower portion relative to the upper portion, and affixes in the lower portion in the realigned position by attaching rigid implants, also known as fixation plates, bone plates or the like, to the lower portion and the upper portion, the fixation implants bridging the osteotomy gap and fastened to the upper and lower portions using mechanical fasteners, i.e., bone screws. The lower portion may be translated, rotated, angled, etc. in one or multiple directions. In the final fixed alignment, the lower portion may be spaced from the upper portion across the osteotomy, such as for example if the lower portion is to be brought forward or down from the upper portion, or a portion of the bone may be removed from the upper and/or lower portion of the maxilla in order to shorten the overall configuration of the post-operative maxilla.

More recently, the use of 3-D imagery such as X-ray or CT-scanning, is utilized in the procedure to better ascertain the desired post-operative configuration and to provide a method for producing customized fixation implants. A pre-operative 3-D image of the maxillary bone is produced showing the current configuration of the bone structure before corrective surgery and the surgeon, along with technicians using dedicated software, is able to perform a virtual osteotomy and then virtually manipulate the free lower portion of the maxilla relative to the fixed upper portion to produce a virtual image of the desired post-operative configuration, location and orientation for the corrected bone structure. Customized virtual implants may then be created that are shaped and configured to match the surface configuration of the maxilla over the areas where the implant will contact the bone surface. Likewise, the portions of the customized virtual implants extending across the osteotomy may be shaped and configured such that once the implant is affixed to the lower and upper portions of the maxilla, the lower portion resides in the desired realigned position relative to the upper portion. An actual implant for use in surgery is then manufactured to match the specifications of the virtual implant.

Under the current systems proper placement of the fixation implants is often difficult, given the variations and contours of the surface configuration for the upper and lower maxilla sections to which the implant is to be attached. It is an object of this invention to provide an improved maxillofacial orthognathic method and related fixation implants, the implants comprising registration members configured to abut, surround and/or engage with the lower regions of the anterior nares (the main anterior nasal opening) and the anterior nasal spine (the prominent process extending anteriorly below the anterior nares), such that proper registration of the implant relative to the upper and lower maxilla sections is readily accomplished.

### BRIEF DESCRIPTION OF THE DRAWINGS

FIGS. 1 and 2 illustrate two embodiments of the implant showing the registration member as a U-shaped member surrounding the anterior nasal spine from below and partially extending into the anterior nares.

FIGS. 3 and 4 illustrate two embodiments of the implant comprising a dental splint and showing the registration member as a U-shaped member surrounding the anterior nasal spine and partially extending into the anterior nares.

FIGS. 5 and 6 illustrate two embodiments of the implant showing the registration member as a circular member surrounding the anterior nasal spine and partially extending into the anterior nares.

FIG. 7 illustrates an embodiment of the implant showing the registration member as a U-shaped member surrounding the anterior nasal spine from above and partially extending into the anterior nares.

### SUMMARY OF THE INVENTION

The invention is a maxillofacial fixation implant having a registration structure or member adapted and structured so as to be positioned to abut, surround and/or engage with the anterior nares and the anterior nasal spine of a human skull, and the method of utilizing the implant to fix separated segments of the maxilla resulting from trauma, malformation and/or orthognathic procedures. For example, in orthognathic procedures, the implant may comprise a pair or set of upper anchor implant portions adapted for affixation to the upper portion of a maxilla and a pair or set of lower anchor implant portions adapted for affixation to the lower portion of a maxilla, the lower portion of the maxilla having been separated by an osteotomy from the upper portion of the maxilla.

The size and 3-D configuration of the implant is determined using 3-D imaging to create the fixation implant such that the lower and upper anchor implant portions conform to the surface configuration of the maxilla, and to create a central registration member conforming to the surface configuration in the lower area of the anterior nares and/or the anterior nasal spine. The registration member may be substantially U-shaped or substantially circular. Mounting apertures to receive fixation screws are provided at locations on the lower and upper anchor implant portions. Preferably no apertures, or only a small number of apertures, are located on the registration member.

### DETAILED DESCRIPTION OF THE INVENTION

The invention in various embodiments is a maxillofacial orthognathic fixation implant 10 having a registration structure or member 13 adapted and structured to be positioned to abut, surround and/or engage with the anterior nares 31 and/or the anterior nasal spine 32 of a human skull, and the method of designing, manufacturing and utilizing the implant 10, wherein the implant 10 comprises a pair of upper anchor implant portions 12 adapted for affixation to the upper portion 33 of a maxilla and a pair of lower anchor implant portions 11 adapted for affixation to the lower portion 34 of a maxilla, the lower portion 34 of the maxilla having been separated by an osteotomy 35 from the upper portion 33 of the maxilla.

The size and 3-D configuration of the implant 10 is determined using 3-D imaging to create the fixation implant such that the inner surface of the lower and the upper anchor implant portions 11/12 conform to the surface configuration of the maxilla over the contact area, and to create a central registration member 13 having an inner surface conforming to the surface bone configuration in the lower area of the anterior nares 31 and the anterior nasal spine 32. In this manner the inner surface of the implant 10 will substantially mate with or match the surface configuration of the bone on which it is positioned. The registration member 13 may be substantially U-shaped or substantially circular. Mounting apertures 14 to receive fixation screws are provided at locations on the lower and upper anchor implant portions 11/12. Preferably, no such apertures 14, or only a minimal number, are located on the registration member 13, as providing apertures 14 on the registration member 13 are typically not required.

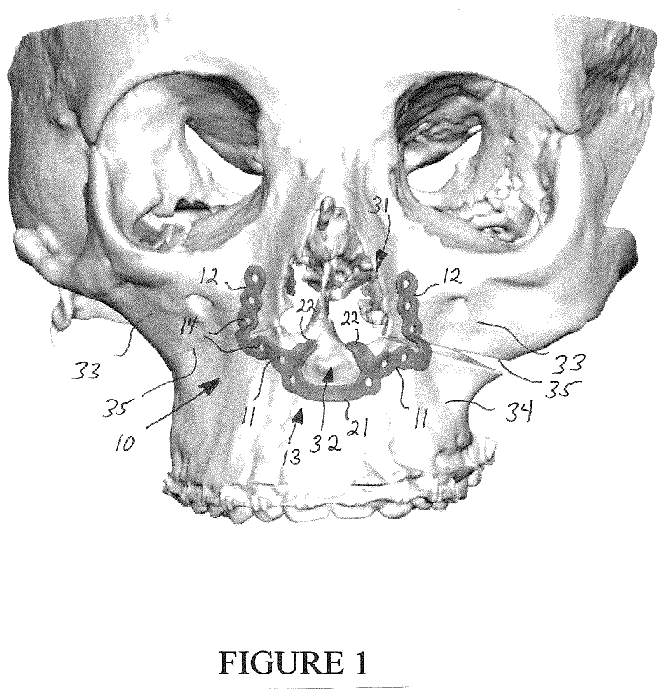

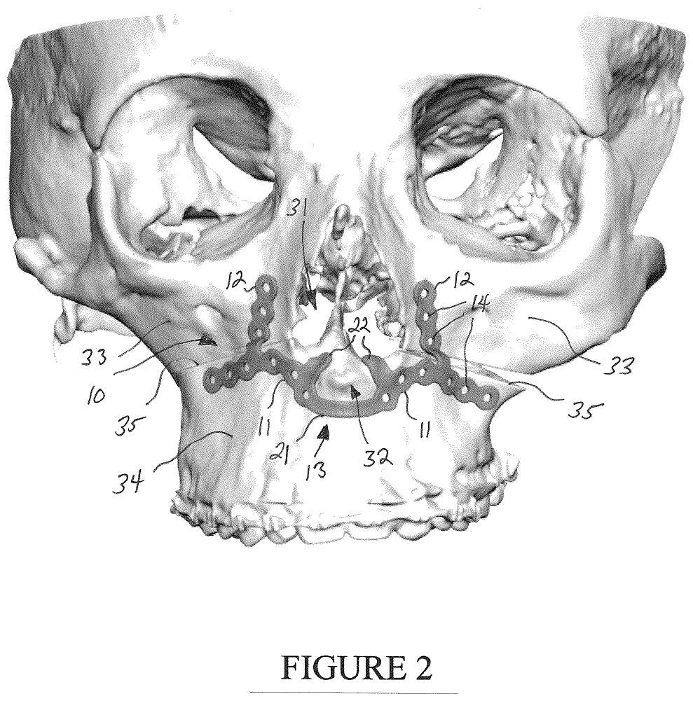

A first set of embodiments is shown in FIGS. 1 and 2, wherein the fixation implant 10 is shown disposed on a human skull, the lower portion 34 of the maxilla having been separated from the upper portion 33 of the maxilla by osteotomies 35. The implant 10 comprises a pair or set of lower anchor implant portions 11 structured, configured and adapted to be affixed onto the maxilla lower portion 34 by fixation screws (not shown) through apertures 14 and a pair or set of upper anchor implant portions 12 connected to the lower anchor implant portions 11 and structured, configured and adapted to be affixed onto the maxilla upper portion 33 by fixation screws (not shown) through apertures 14. Critically, the implant 10 further comprises a centrally or medially located registration member 13, the registration member located between and connecting the two lower anchor implant portions 11.

The registration member 13 of the embodiments shown in FIGS. 1 and 2 is generally or substantially U-shaped in configuration, comprising a lower transverse portion 21 and two upper end portions 22. The registration member 13 is adapted, configured and structured to abut or partially surround the anterior nasal spine 32. The lower transverse portion 21 of the registration member 13 is adapted, configured and structured to be positioned beneath the anterior nasal spine 32 and the upper end portions 22 are adapted, configured and structured to be positioned one to each side of the anterior nasal spine 32, such that the upper end portions 22 preferably extend a short distance into the anterior nares 31 so as to rest on the bone structure defining the lower part of the anterior nares 31. The upper end portions 22 may be joined directly to the lower transverse portion 21 joined to the lower transverse portion 21 or in combination with end segments of the lower anchor implant portions 11.

Because the implant 10 is particularized for a particular patient with computer aided custom design, the overall configuration and contouring of the implant 10, and especially its interior surface, directly conforms, matches or mates with the surface configuration and contour of the maxilla over the areas of direct contact. Proper positioning of the implant 10 prior to affixation to the maxilla is easily accomplished by matching the medially located registration member 13 to the maxilla or skull area adjacent the anterior nasal spine 32 and anterior nares 31. Improper relative positioning will be readily noticed since the implant 11 will not rest on the maxilla in a nested or stable manner.

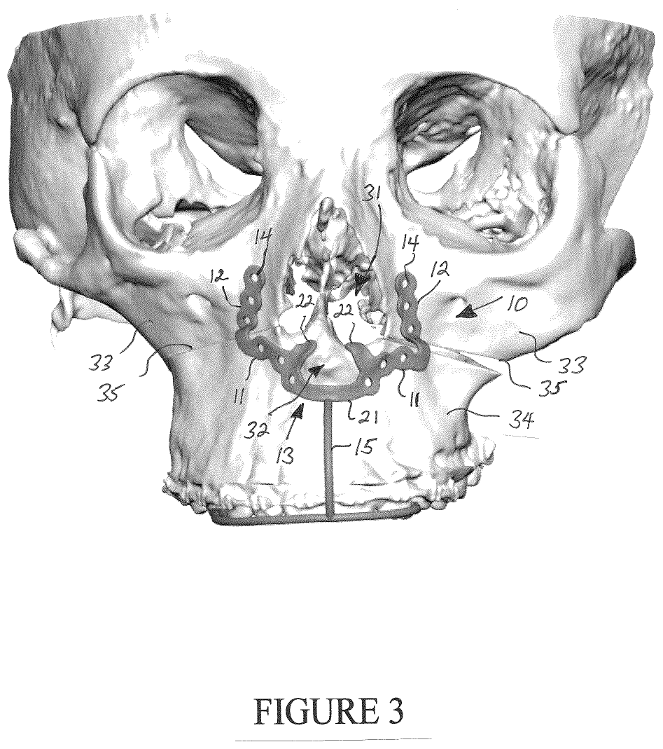

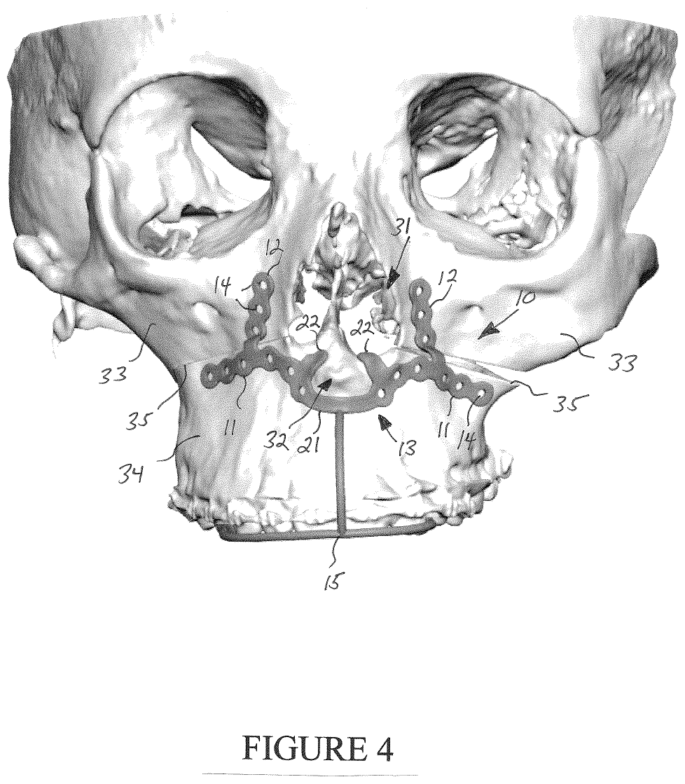

FIGS. 3 and 4 illustrate alternative embodiments of the implant 10 wherein the implant 10 further comprises a dental splint connected to the lower transverse portion 21 of the registration member 13.

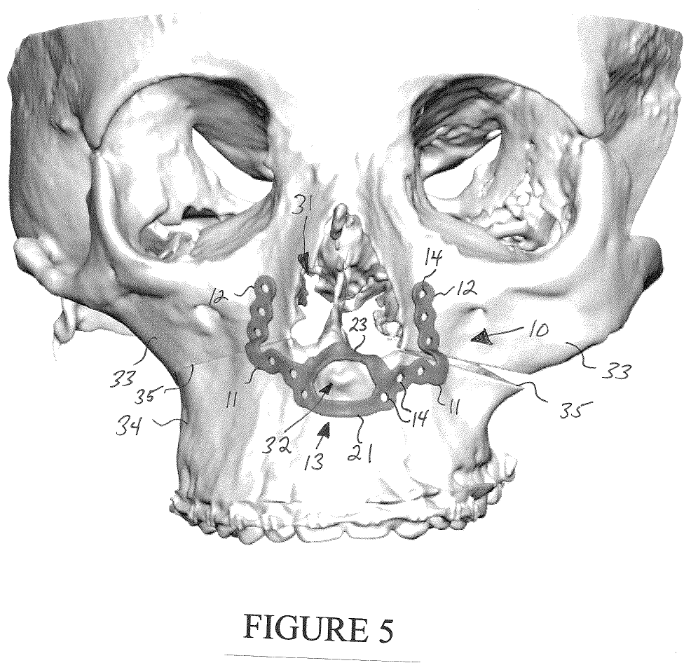

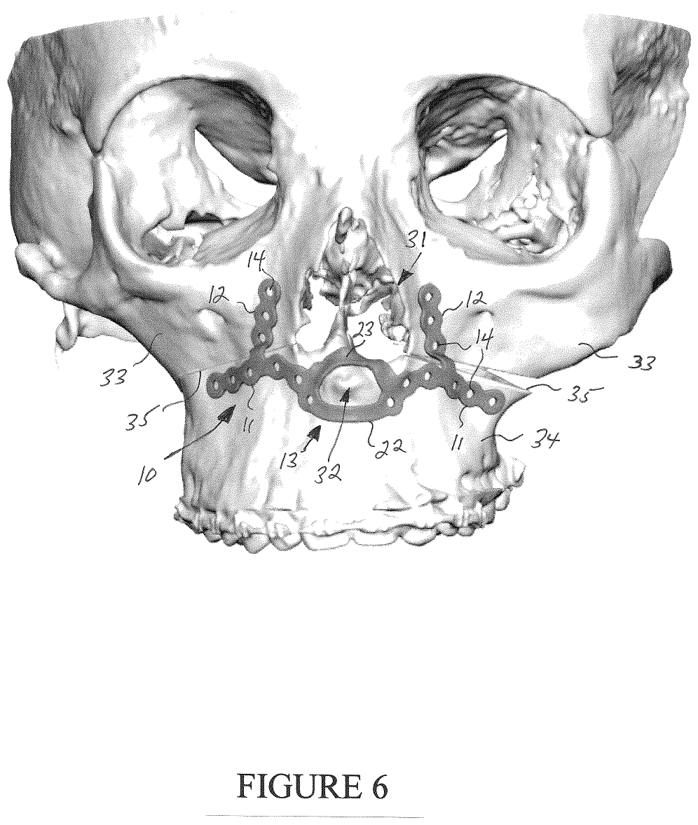

FIGS. 5 and 6 illustrate alternative embodiments of the implant 10 wherein the registration member 13 is a generally or substantially circular member formed by the combination of a lower transverse member 21 joined to an upper transverse portion 23 such that no ends are present, i.e., the registration member 13 is either a continuous member or the lower and upper transverse portions 21/23 are joined by combining with short end segments of the lower anchor implant portions 11. The lower transverse member 21 as before resides below the anterior nasal spine 32 while the upper transverse portion 23 resides above the anterior nasal spine 32. Preferably, portions of the upper transverse portion 23, formed such as folds or short tabs, extend into the anterior nares 31 so as to rest on the bone structure defining the lower part of the anterior nares 31. In this manner the anterior nasal spine is surrounded by the registration member 13.

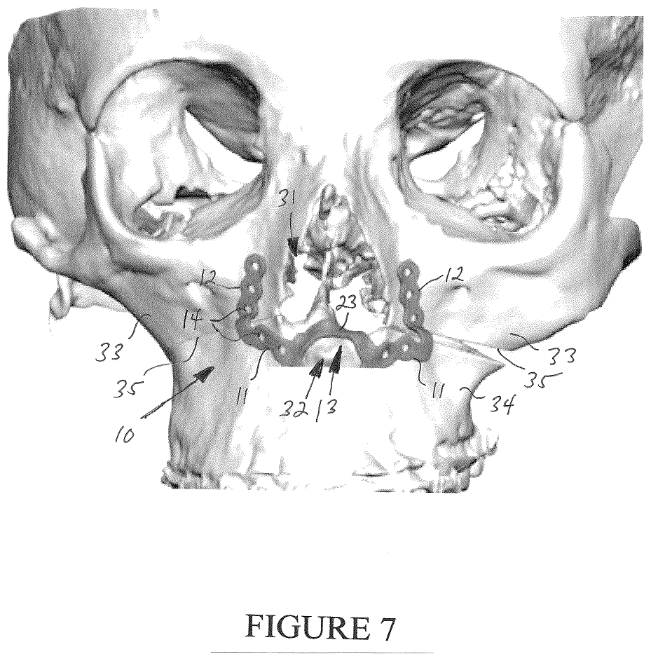

FIG. 7 illustrates an alternative embodiment of the implant 10 wherein the implant 10 comprises a pair of lower anchor implant portions 11, which will be affixed to the maxilla lower portion 34 by fixation screws (not shown) through apertures 14, and upper anchor implant portions 12 foined to the lower anchor implant portions 11, which will be affixed to the maxilla upper portion 33 by fixation screws (not shown) through apertures 14. The implant 10 further comprises a centrally or medially located registration member 13, the registration member 13 joined to and connecting the two lower anchor implant portions 11.

The registration member 13 is generally or substantially U-shaped in configuration and comprises an upper transverse portion 23. The registration member 13 is configured to abut or partially surround the anterior nasal spine 32, the upper transverse portion 23 being positioned above the anterior nasal spine 32, and is preferably configured such that one or more portions of the upper transverse portion 23, such as in the form of folds or tabs, extend or overlap over a short distance into the anterior nares 31 so as to rest on the bone structure defining the lower part of the anterior nares 31.

It is understood that equivalents and substitutions for certain elements described above may be obvious to those of skill in the art, and therefore the true scope and definition of the invention is to be as set forth in the following claims.

I claim:

1. A maxillofacial orthognathic fixation implant positioning method, the method configured to properly position a maxillofacial orthognathic fixation implant onto a human skull having an anterior nares, an anterior nasal spine and a maxilla, each having a surface configuration, wherein the maxilla is divided by osteotomies into an upper maxilla portion and a lower maxilla portion, the method comprising the steps of:
creating a virtual pre-operative 3-D image of the anterior nares, anterior nasal spine and maxilla of a human skull through CT scanning or similar imaging methodologies;
defining a virtual osteotomy of the virtual maxilla to virtually create a virtual free portion of the virtual maxilla and a virtual fixed portion of the virtual maxilla;
virtually repositioning the virtual free portion of the virtual maxilla to a desired post-operative location and orientation relative to the virtual fixed portion of the virtual maxilla;
creating a virtual maxillofacial orthognathic fixation implant configured to match the surface configuration of the virtual maxilla where the virtual maxillofacial orthognathic fixation implant makes contact with the surface configuration of the virtual maxilla;
wherein the step of creating a virtual maxillofacial orthognathic fixation implant includes the step of creating virtual upper anchor implant portions configured to be positioned onto the virtual fixed portion of the virtual maxilla and virtual lower anchor implant portions configured to be positioned onto the virtual fixed portion of the virtual maxilla, wherein the virtual lower anchor implant portions are joined to the virtual upper anchor implant portions;
wherein the step of creating a virtual maxillofacial orthognathic fixation implant includes the step of creating and adding one or more virtual central registration members to the virtual maxillofacial orthognathic fixation implant such that the one or more virtual central registration members virtually abut, surround and/or engage the virtual anterior nares and/or virtual anterior nasal spine of the virtual human skull;
creating a maxillofacial orthognathic fixation implant matching the virtual maxillofacial orthognathic fixation implant such that the maxillofacial orthognathic fixation implant comprises one or more central registration members;
properly positioning the maxillofacial orthognathic fixation implant on the human skull by orienting the one or more central registration members to abut, surround and/or engage the anterior nares and/or anterior nasal spine of the human skull.

2. The method of claim 1, wherein the step of creating and adding one or more virtual central registration members to the virtual maxillofacial orthognathic fixation implant further comprises configuring the one or more virtual central registration members to be U-shaped and comprise a pair of virtual upper end portions connected by a virtual lower transverse portion.

3. The method of claim 2, wherein the step of creating and adding one or more virtual central registration members to the virtual maxillofacial orthognathic fixation implant further comprises configuring the one or more U-shaped virtual central registration members to comprise a pair of virtual upper end portions connected by a virtual lower transverse portion, wherein the pair of virtual upper end portions are configured to extend into the virtual anterior nares.

4. The method of claim 1, wherein the step of creating a virtual maxillofacial orthognathic fixation implant further includes creating a virtual dental split connected to the virtual registration member.

5. The method of claim 1, wherein the step of creating and adding one or more virtual central registration members to the virtual maxillofacial orthognathic fixation implant further comprises configuring the one or more virtual central registration members to be circular and comprise a virtual lower transverse member and a virtual upper transverse member.

6. The method of claim 5, wherein the step of creating and adding one or more virtual central registration members to the virtual maxillofacial orthognathic fixation implant further comprises configuring the virtual upper transverse member of the one or more circular virtual central registration members to extend into the virtual anterior nares.

7. The method of claim 1, wherein the step of creating and adding one or more virtual central registration members to the virtual maxillofacial orthognathic fixation implant further comprises configuring the one or more virtual central registration members to be U-shaped and comprise a virtual upper transverse portion.

8. The method of claim 7, wherein the step of creating and adding one or more virtual central registration members to the virtual maxillofacial orthognathic fixation implant further comprises configuring the virtual upper transverse portion of the one or more U-shaped virtual central registration members to extend into the virtual anterior nares.

9. The method of claim 1, wherein the step of creating virtual upper anchor implant portions configured to be positioned onto the virtual fixed portion of the virtual maxilla and virtual lower anchor implant portions configured to be positioned onto the virtual fixed portion of the virtual maxilla further includes the step of providing virtual apertures in the virtual upper implant anchor portions and the virtual lower implant anchor portions and not providing apertures in the virtual central registration members.

10. The method of claim 1, wherein the step of creating virtual upper anchor implant portions configured to be positioned onto the virtual fixed portion of the virtual maxilla and virtual lower anchor implant portions configured to be positioned onto the virtual fixed portion of the virtual maxilla further includes the step of providing virtual apertures in the virtual upper implant anchor portions, the virtual lower implant anchor portions and the virtual central registration members.
